(19) United States
(12) Patent Application Publication
(10) Pub. No.: US 2019/0090851 A1
(43) Pub. Date: Mar. 28, 2019

(54) ULTRASONIC DIAGNOSTIC APPARATUS AND PROGRAM

(71) Applicant: General Electric Company, Schenectady, NY (US)

(72) Inventors: Atsuko Matsunaga, Tokyo (JP); Shunichiro Tanigawa, Tokyo (JP)

(21) Appl. No.: 15/528,797

(22) PCT Filed: Nov. 26, 2014
(86) PCT No.: PCT/US2015/062527

§ 371 Date: May 23, 2017

(30) Foreign Application Priority Data
Nov. 26, 2014 (JP) 2014-238531

(51) Int. Cl. A61B 8/08; A61B 8/00; A61B 8/14

(52) U.S. Cl. CPC A61B 8/485; A61B 8/463; A61B 8/54; A61B 8/469; A61B 8/14; A61B 8/5246; A61B 8/4455; A61B 8/5223

(57) ABSTRACT

An ultrasonic diagnostic apparatus includes: an ultrasonic probe for transmitting a first ultrasonic beam BM1 to biological tissue in a subject; a transmission control section for transmitting an ultrasonic beam for generating shear waves in the biological tissue from the ultrasonic probe to the biological tissue while applying steering to the beam; and a region-defining section for defining a region in an ultrasonic image of the subject, wherein the transmission control section transmits the first ultrasonic beam while applying steering to the beam by setting transmission parameters such that the first ultrasonic beam travels closest to and outside of the region.

### CROSS-REFERENCE TO RELATED APPLICATIONS

This application is a filing under 35 U.S.C. 371 of international application number PCT/US2015/062527, filed Nov. 24, 2015, which claims priority to Japanese application number 2014-238531, filed Nov. 26, 2014, the entire disclosure of each of which is hereby incorporated by reference.

### FIELD OF THE INVENTION

The present invention relates to an ultrasonic diagnostic apparatus and a program for measuring elasticity of biological tissue by transmitting an ultrasonic beam having a high acoustic pressure from an ultrasonic probe.

### BACKGROUND

There have been known elasticity measurement techniques of measuring elasticity of biological tissue by transmitting an ultrasonic beam (push pulse) having a high acoustic pressure from an ultrasonic probe to the biological tissue (for example, see Patent Document 1). More particularly, shear waves generated in the biological tissue by the ultrasonic beam are detected by ultrasonic detecting beams, and the velocity of propagation of the shear waves and/or the elasticity value of the biological tissue are calculated to provide elasticity data. Then, an elasticity image having colors or the like depending upon the elasticity data is displayed in a two-dimensional region.

The shear waves attenuate as they travel farther away from the ultrasonic beam from which they are generated. Therefore, in the case that the ultrasonic beam is transmitted to a position away from the region described above, it is difficult to provide an elasticity value accurately reflecting elasticity of biological tissue because of a smaller amplitude of the shear waves detected in the inside of the region. Moreover, the ultrasonic beam for generating shear waves transmitted to a position away from the region may increase the likelihood that an obstacle hampering propagation of the shear waves into the region be present between the ultrasonic beam and the region. Thus, it is desirable to transmit the ultrasonic beam to a position as close to the region as possible.

Generally, the beam direction of an ultrasonic beam is perpendicular to a transmission/reception plane for an ultrasonic probe. Thus, the ultrasonic beam may be transmitted to a position away from the region depending upon the geometry of the ultrasonic probe. In particular, a convex probe having a curved transmission/reception plane may cause the ultrasonic beam to be transmitted to a position away from a defined region depending upon the position of the region. Consequently, the shear waves detected in the inside of the region may have lower intensity or may be hampered from propagating to the region. Accordingly, there is a need for an ultrasonic diagnostic apparatus and a program capable of more reliably propagating the shear waves to the region while suppressing attenuation regardless of the geometry of an ultrasonic probe.

### BRIEF DESCRIPTION

The invention, in one aspect, made for solving the problem described above is an ultrasonic diagnostic apparatus, including: an ultrasonic probe for transmitting an ultrasonic beam to biological tissue in a subject; a transmission control section for transmitting an ultrasonic beam for generating shear waves in said biological tissue from said ultrasonic probe to said biological tissue while applying steering to said beam; and a region-defining section for defining a region in an ultrasonic image of said subject, wherein said transmission control section transmits said ultrasonic beam while applying steering to said beam by setting transmission parameters such that said ultrasonic beam travels closest to and outside of said region.

The invention, in another aspect, made for solving the problem described above is an ultrasonic diagnostic apparatus, including: an ultrasonic probe for transmitting an ultrasonic beam to biological tissue in a subject; a transmission control section for transmitting an ultrasonic beam for generating shear waves in said biological tissue from said ultrasonic probe to said biological tissue while applying steering to said beam; and a region-defining section for defining a region in an ultrasonic image of said subject, wherein said transmission control section transmits a pair of said ultrasonic beams while applying steering to said beams by setting transmission parameters such that said ultrasonic beams are each transmitted to a position that lies in the vicinity of either end of said region in a lateral direction and causes at least part of said ultrasonic beams to be included in said region.

According to the invention in the one aspect, the transmission control section transmits the ultrasonic beam while applying steering to the beam by setting transmission parameters such that the ultrasonic beam travels closest to and outside of the region, and therefore, shear waves generated by the ultrasonic beam may be more reliably propagated to the region while suppressing attenuation.

According to the invention in the other aspect, the transmission control section transmits a pair of the ultrasonic beams while applying steering to the beams by setting transmission parameters such that the ultrasonic beams are each transmitted to a position that lies in the vicinity of either end of the region in a lateral direction and causes at least part of the ultrasonic beams to be included in the region, shear waves generated by the ultrasonic beams may be more reliably propagated to the region while suppressing attenuation.

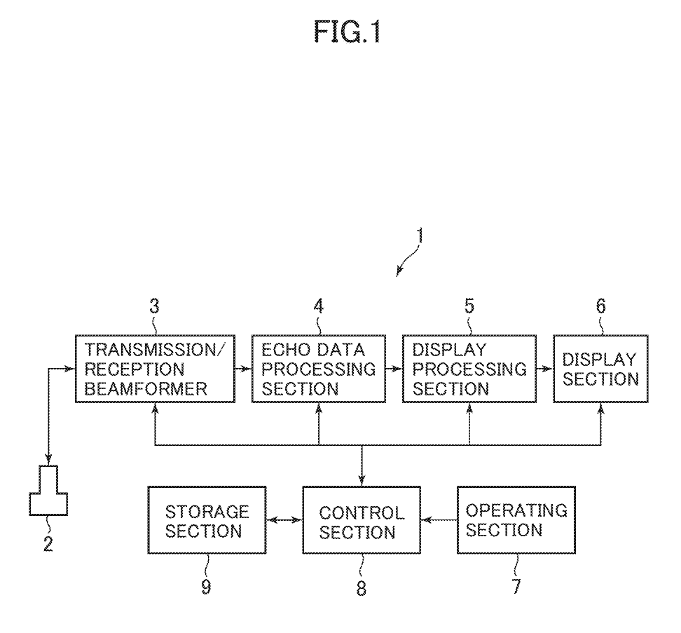

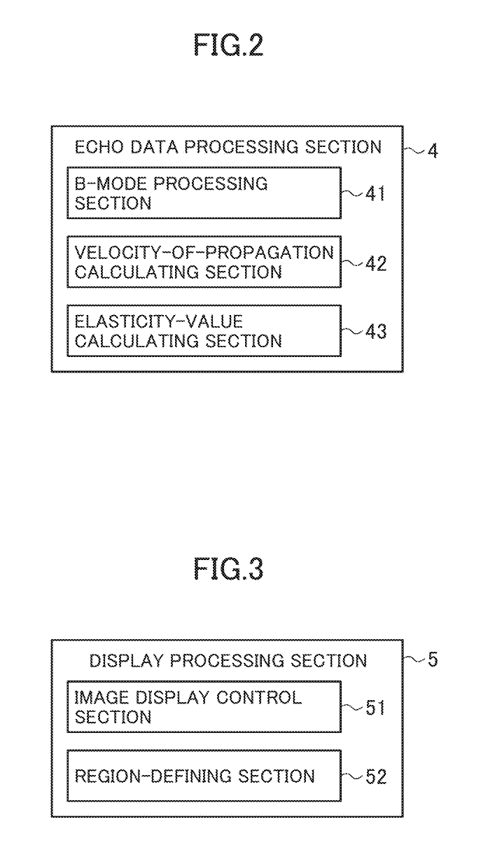

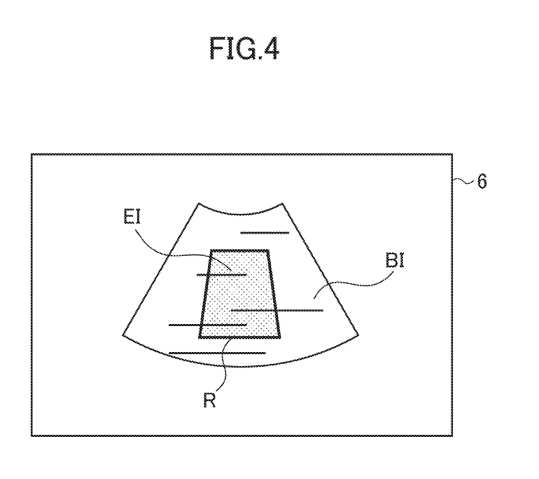

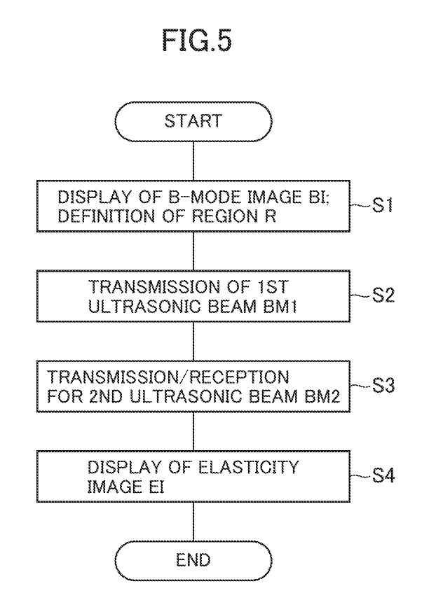

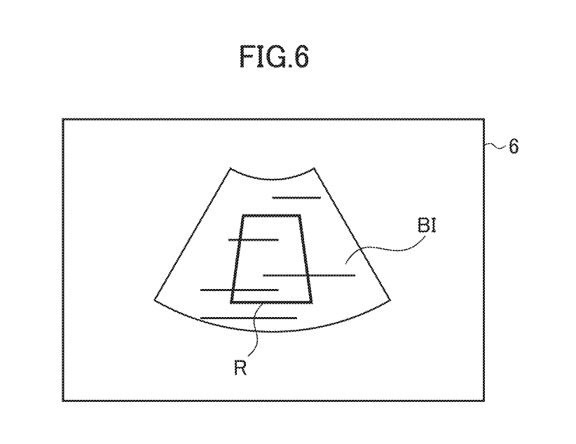

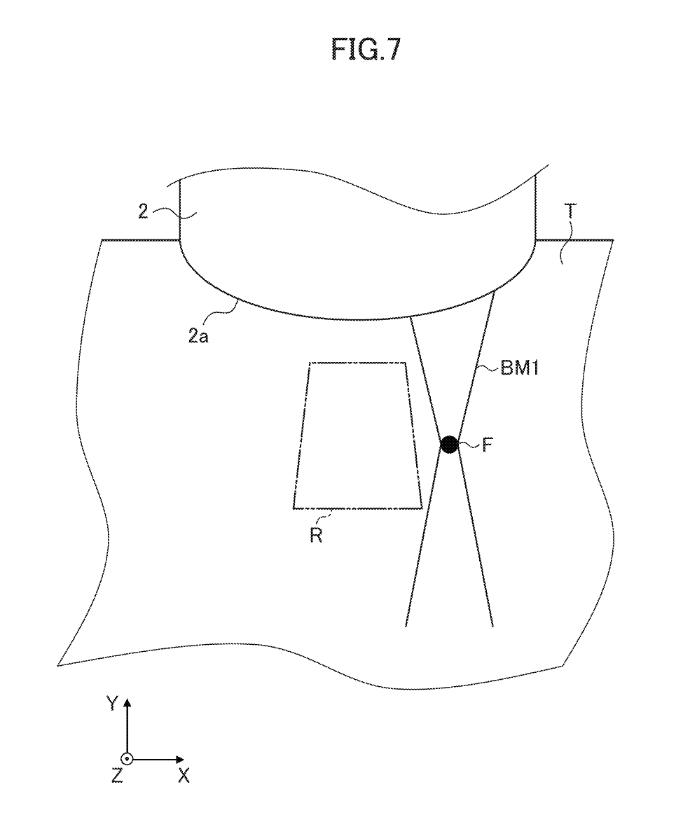

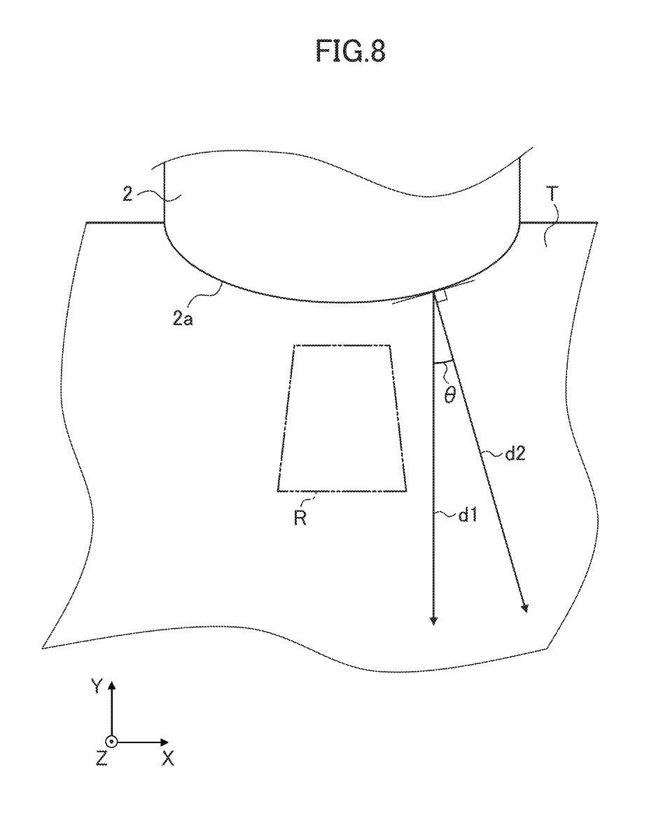

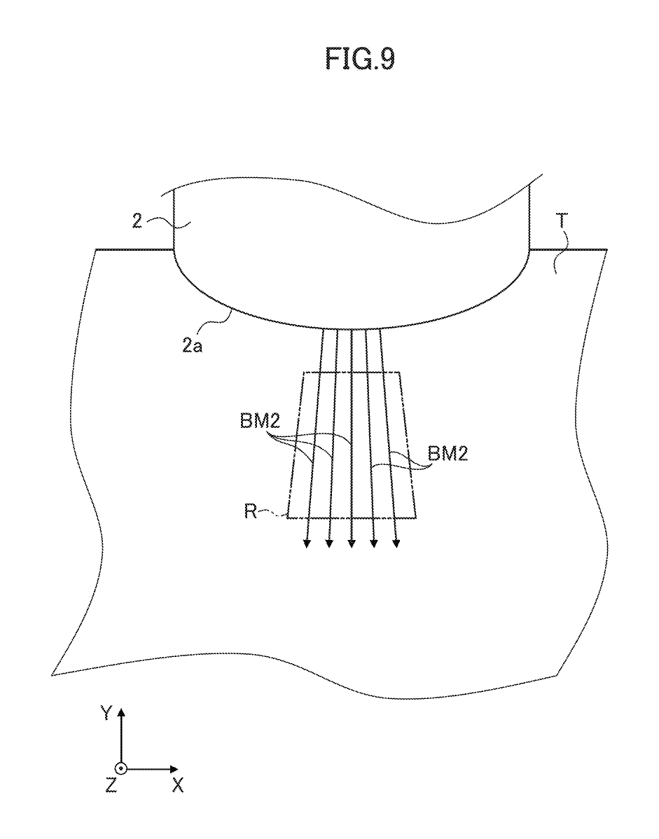

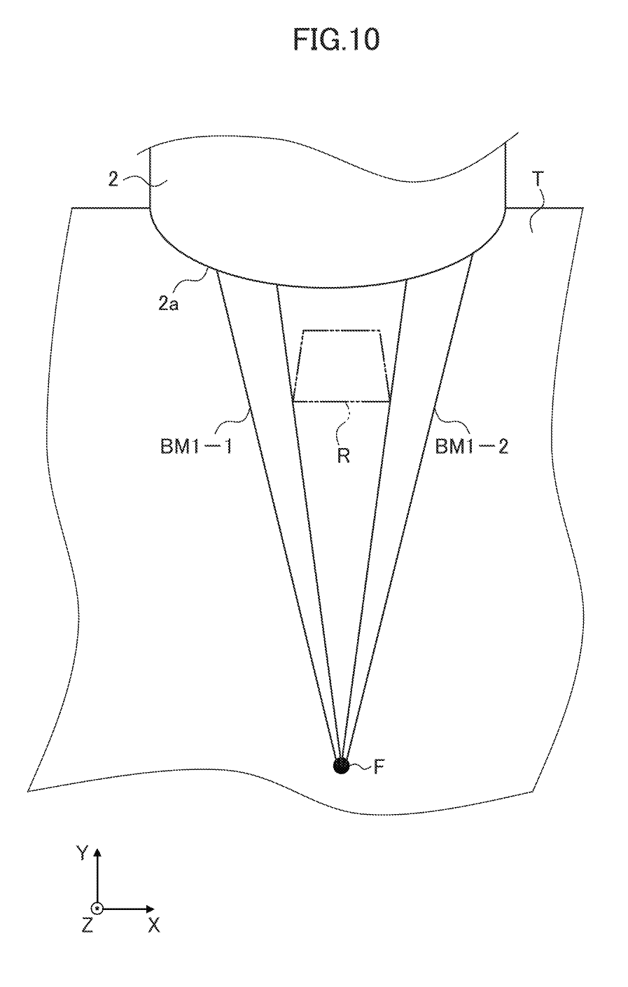

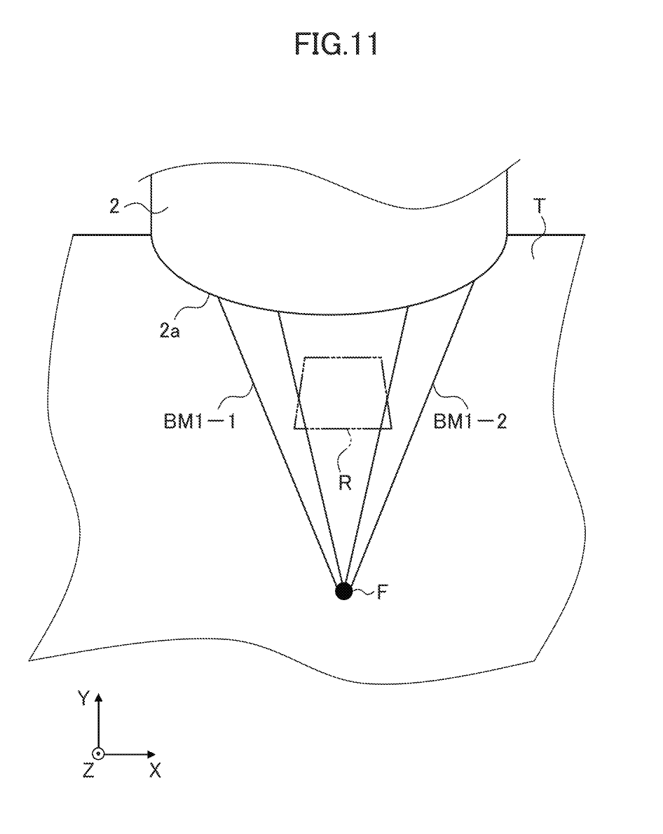

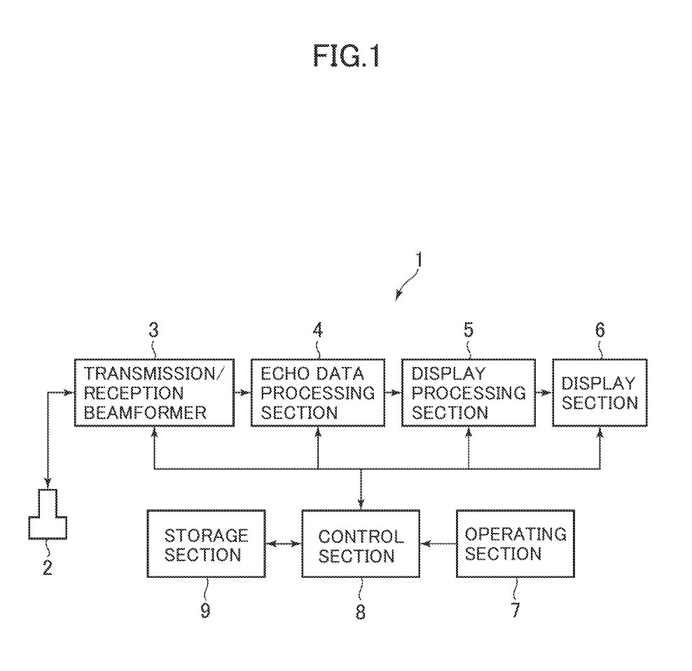

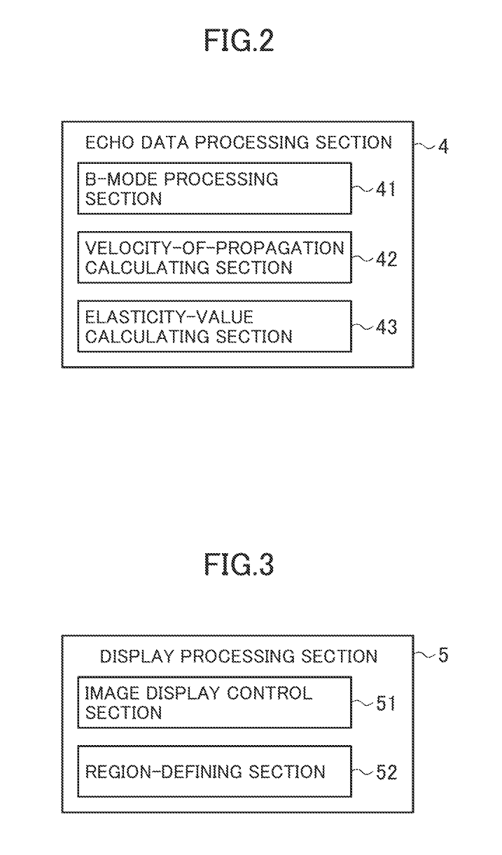

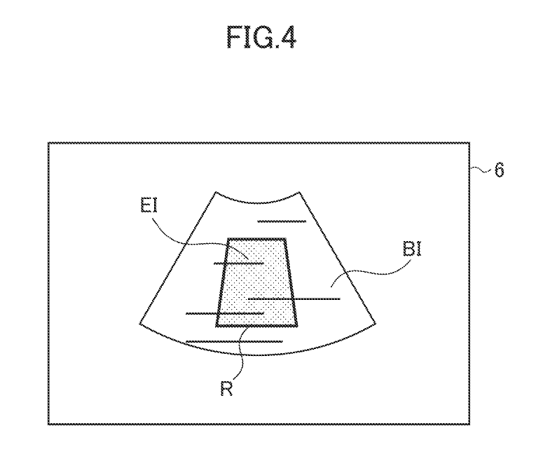

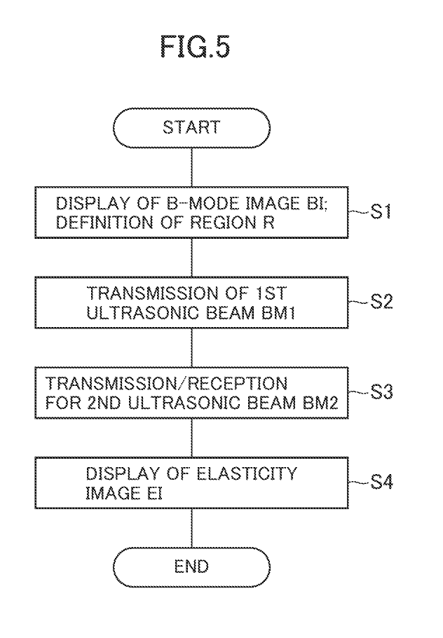

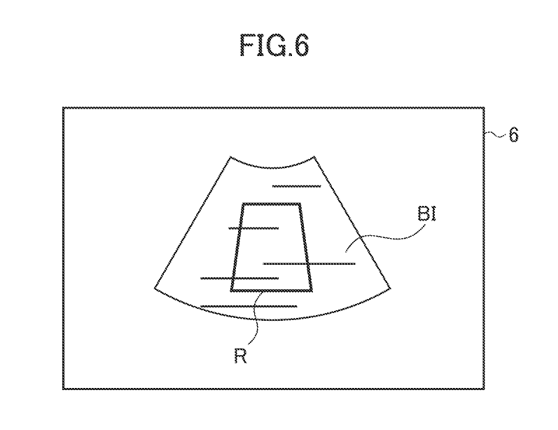

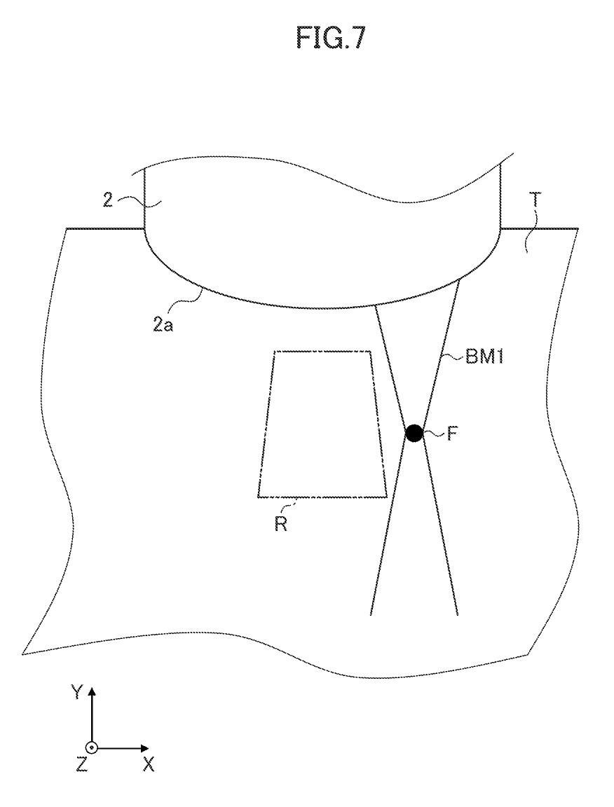

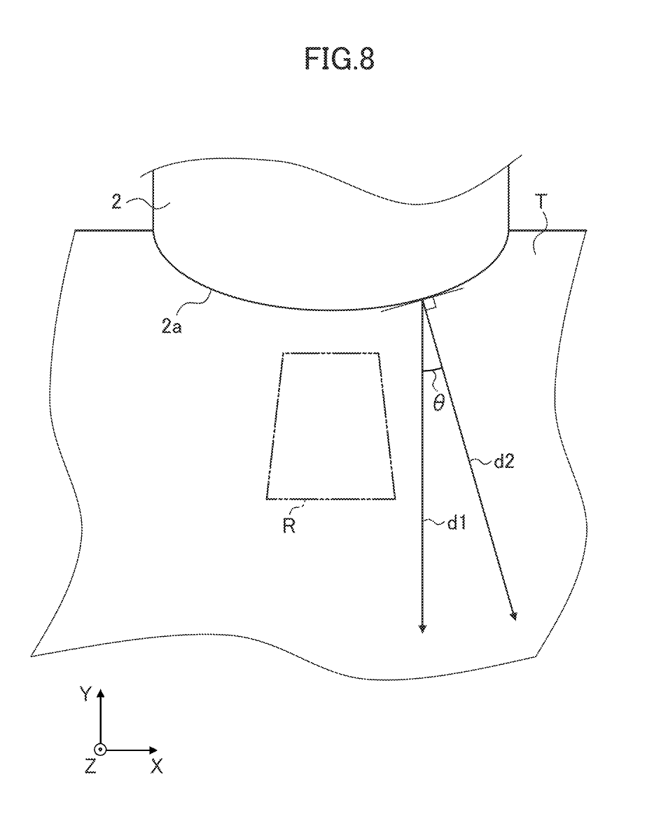

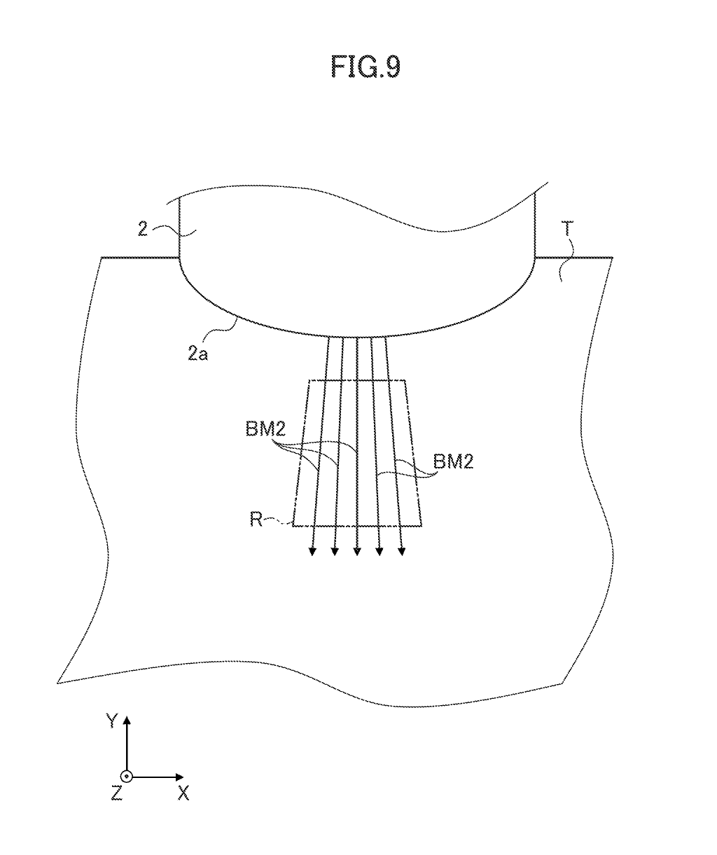

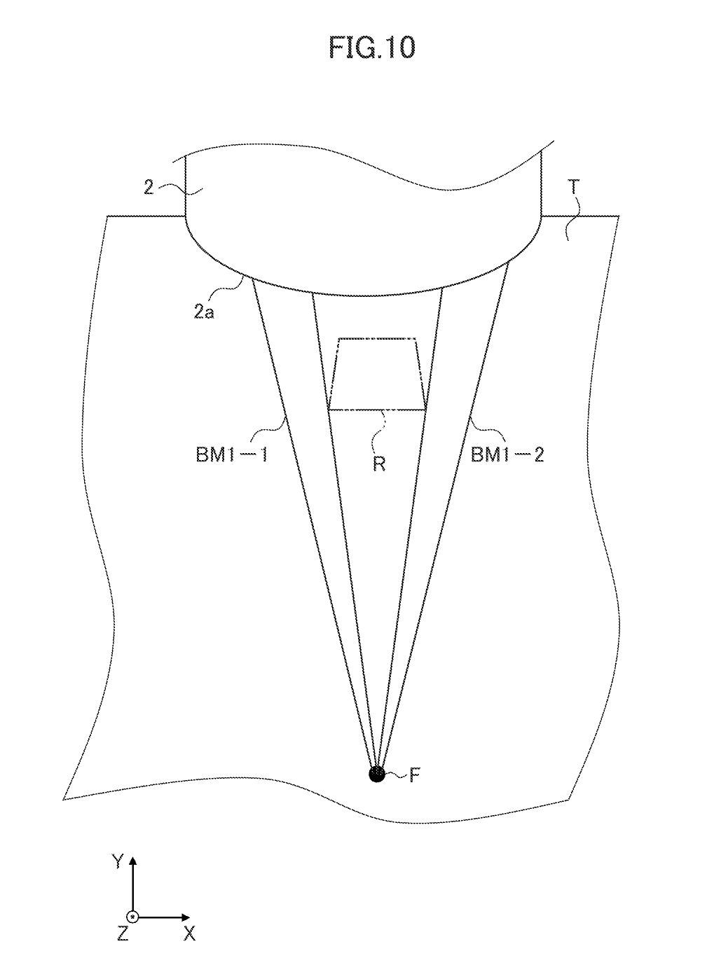

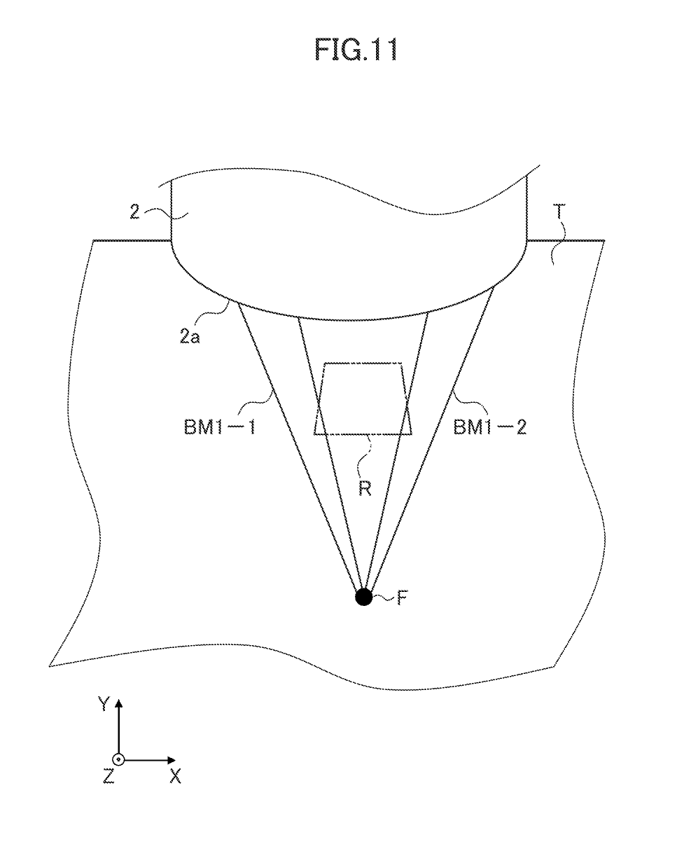

### BRIEF DESCRIPTION OF THE DRAWINGS

[FIG. 1] A block diagram showing a schematic configuration of an ultrasonic diagnostic apparatus, which is an exemplary embodiment of the present invention.

[FIG. 2] A block diagram showing a configuration of an echo data processing section.

[FIG. 3] A block diagram showing a configuration of a display processing section.

[FIG. 4] A diagram showing a display section in which a B-mode image and an elasticity image are displayed.

[FIG. 5] A flow chart showing an operation of a first embodiment.

[FIG. 6] A diagram showing the display section in which a region is defined in the B-mode image.

[FIG. 7] A diagram explaining transmission of a first ultrasonic beam.

[FIG. 8] A diagram explaining a beam direction of the first ultrasonic beam.

[FIG. 9] A diagram explaining a second ultrasonic beam.

[FIG. 10] A diagram explaining transmission of the first ultrasonic beam in a first variation of the embodiment.

[FIG. 11] A diagram explaining transmission of the first ultrasonic beam in a second variation the embodiment.

### DETAILED DESCRIPTION

Now an embodiment of the present invention will be described. An ultrasonic diagnostic apparatus 1 shown in FIG. 1 comprises an ultrasonic probe 2, a transmission/reception (T/R) beamformer 3, an echo data processing section 4, a display processing section 5, a display section 6, an operating section 7, a control section 8, and a storage section 9. The ultrasonic diagnostic apparatus 1 has a configuration as a computer.

The ultrasonic probe 2 is configured to comprise a plurality of ultrasonic vibrators (not shown) arranged in an array, for transmitting an ultrasonic beam (ultrasonic pulse) to a subject and receiving echo signals thereof by the ultrasonic vibrators. In the present embodiment, the ultrasonic probe 2 is a convex probe having a curved plane for transmission/reception of an ultrasonic beam.

By the ultrasonic probe 2, a first ultrasonic beam (push pulse) for generating shear waves in biological tissue is transmitted. Also by the ultrasonic probe 2, a second ultrasonic beam for detecting the shear waves is transmitted and echo signals thereof are received. Moreover, by the ultrasonic probe 2, a third ultrasonic beam for producing a B-mode image is transmitted and echo signals thereof are received.

The T/R beamformer 3 drives the ultrasonic probe 2 based on control signals from the control section 8 to transmit the first through third ultrasonic beams with predetermined transmission parameters (transmission control function). The T/R beamformer 3 also applies signal processing such as phased addition processing to ultrasonic echo signals. The T/R beamformer 3 and control section 8 represent an exemplary embodiment of the transmission control section in the present invention. The transmission control function represents an exemplary embodiment of the transmission control function in the present invention.

The echo data processing section 4 comprises a B-mode processing section 41, a velocity-of-propagation calculating section 42, and an elasticity-value calculating section 43, as shown in FIG. 2. The B-mode processing section 41 applies B-mode processing such as logarithmic compression processing and envelope detection processing to echo data output from the T/R beamformer 3, and creates B-mode data. The B-mode processing section 41 creates the B-mode data based on echo signals of the third ultrasonic beam.

The velocity-of-propagation calculating section 42 calculates a velocity of propagation of the shear waves based on echo data output from the T/R beamformer 3. The velocity-of-propagation calculating section 42 calculates the velocity of propagation based on echo signals of the second ultrasonic beam. The elasticity-value calculating section 43 also calculates an elasticity value of the biological tissue to which a push pulse is transmitted based on the velocity of propagation. Details thereof will be discussed later. The velocity-of-propagation calculating section 42 and elasticity-value calculating section 43 represent an exemplary embodiment of the measurement-value calculating section in the present invention. The velocity of propagation and elasticity value represent an exemplary embodiment of the measurement value regarding elasticity of the biological tissue in the present invention

It should be noted that only the velocity of propagation may be calculated without necessarily calculating the elasticity value. Data of the velocity of propagation or data of the elasticity value will be referred to herein as elasticity data.

The display processing section 5 comprises an image display processing section 51 and a region-defining section 52, as shown in FIG. 3. The image display processing section 51 scan-converts the B-mode data by a scan converter to create B-mode image data, based on which a B-mode image is displayed in the display section 6. The image display processing section 51 also scan-converts the elasticity data by the scan converter to create elasticity image data, based on which an elasticity image is displayed in the display section 6.

Referring to FIG. 4, the elasticity image EI is a two-dimensional image displayed within a two-dimensional region R defined in the B-mode image BI. The elasticity image EI is a color image having colors according to the velocity of propagation or the elasticity value. The image display processing section 51 combines the B-mode image data and elasticity image data together to create combined image data, based on which an image is displayed in the display section 6. Therefore, the elasticity image EI is a semi-transparent image through which the B-mode image BI in the background is allowed to pass.

The B-mode image BI represents an exemplary embodiment of the ultrasonic image in the present invention. The elasticity image EI represents an exemplary embodiment of the elasticity image in the present invention.

The region R is defined by the region-defining section 52. More specifically, the region-defining section 52 defines the region R based on an input by an operator at the operating section 7. The region R is a region in which shear waves are to be detected, and transmission/reception of the second ultrasonic beam is performed in this region R. The region-defining section 52 represents an exemplary embodiment of the region-defining section in the present invention. The function of defining the region R by the region-defining section 52 represents an exemplary embodiment of the region-defining function in the present invention. The region R represents an exemplary embodiment of the region in the present invention.

The display section 6 is an LCD (Liquid Crystal Display), an organic EL (Electro-Luminescence) display, or the like. The display section 6 represents an exemplary embodiment of the display section in the present invention.

The operating section 7 is configured to comprise a keyboard for allowing an operator to input a command and/or information, a pointing device such as a trackball, and the like, although not particularly shown.

The control section 8 is a processor such as a CPU (Central Processing Unit). The control section 8 loads thereon a program stored in the storage section 9 and controls several sections in the ultrasonic diagnostic apparatus 1. For example, the control section 8 loads thereon a program stored in the storage section 9 and executes functions of the T/R beamformer 3, echo data processing section 4, and display processing section 5 by the loaded program.

The control section 8 may execute all of the functions of the T/R beamformer 3, all of the functions of the echo data processing section 4, and all of the functions of the display processing section 5 by the program, or execute only some of the functions by the program. In the case that the control section 8 executes only some of the functions, the remaining functions may be executed by hardware such as circuitry.

It should be noted that the functions of the T/R beamformer 3, echo data processing section 4, and display processing section 5 may be implemented by hardware such as circuitry.

The storage section 9 is an HDD (Hard Disk Drive), semiconductor memory such as RAM (Random Access Memory) and/or ROM (Read-Only Memory), and the like. The ultrasonic diagnostic apparatus 1 may have all of the HDD, RAM, and ROM as the storage section 9. The storage section 9 may also be any portable storage medium such as a CD (Compact Disk) or a DVD (Digital Versatile Disk).

The program executed by the control section 8 is stored in a non-transitory storage medium such as an HDD or ROM. The program may also be stored in any non-transitory portable storage medium such as a CD (Compact Disk) or a DVD (Digital Versatile Disk).

Next, an operation of the ultrasonic diagnostic apparatus 1 in the present embodiment will be described based on the flow chart in FIG. 5. First, at Step S1, an operator performs ultrasound transmission/reception to/from biological tissue in a subject by the ultrasonic probe 2, and displays a B-mode image BI based on echo signals. At Step S1, a third ultrasonic beam is transmitted. The third ultrasonic beam represents an exemplary embodiment of the ultrasonic beam that is transmitted separately from the ultrasonic beam for generating shear waves in the present invention.

The operator then makes an input for defining a region R in the B-mode image BI at the operating section 7. Thus, the region R is defined in the B-mode image BI, as shown in FIG. 6. The region R is defined to have a position and a size in which the operator desires to display an elasticity image.

Next, at Step S2, one aforementioned first ultrasonic beam BM1 is transmitted from the ultrasonic probe 2 to biological tissue T, as shown in FIG. 7. The first ultrasonic beam BM1 is transmitted as soon as the operator has made an input at the operating section 7 for displaying an elasticity image, for example. The first ultrasonic beam BM1 is transmitted to the outside of the region R and in the vicinity of one end of the region R in a lateral direction (X direction). The first ultrasonic beam BM1 is an ultrasonic beam for generating shear waves in the biological tissue, and represents an exemplary embodiment of the ultrasonic beam for generating shear waves in the present invention.

Now the first ultrasonic beam BM1 will be described in detail. The T/R beamformer 3 sets transmission parameters such that the first ultrasonic beam BM1 travels closest to and outside of the region R, and transmits the first ultrasonic beam BM1. More particularly, the T/R beamformer 3 transmits the first ultrasonic beam BM1 to the biological tissue while applying steering to the beam. In other words, the T/R beamformer 3 transmits the first ultrasonic beam BM1 in a direction d1 at a predefined angle θ(θ≠0) with respect to a direction d2 orthogonal to a tangential direction of a transmission/reception plane 2a in the ultrasonic probe 2, as shown in FIG. 8. The direction d2 is a beam direction when no steering is applied to the ultrasonic beam.

Moreover, the T/R beamformer 3 transmits the first ultrasonic beam BM1 having a focus F with prespecified depth, as shown in FIG. 7 described above.

The T/R beamformer 3 adjusts the direction and shape of the first ultrasonic beam BM1 by setting the transmission parameters such as the amount of delay, transmission aperture, focus, etc. such that the first ultrasonic beam BM1 travels closest to the region R based on information on the position (position and size) of the region R.

The first ultrasonic beam BM1 generates shear waves in the biological tissue T. At Step S3, a second ultrasonic beam BM2 for detecting shear waves generated in the inside of the region R by the first ultrasonic beam BM1 is transmitted, and echo signals thereof are received, as shown in FIG. 9. It should be noted that the second ultrasonic beam BM2 is indicated by acoustic lines in FIG. 9. The transmission/reception of the second ultrasonic beam BM2 is sequentially performed for a plurality of acoustic lines in the inside of the region R.

Next, at Step S4, elasticity data is created based on the echo signals of the second ultrasonic beam BM2, and an elasticity image EI based on the elasticity data is displayed. The elasticity data is data of the velocity of propagation of the shear waves or data of an elasticity value calculated based on the velocity of propagation. More specifically, the velocity-of-propagation calculating section 42 calculates a velocity of propagation of the shear waves detected in the echo signals of the second ultrasonic beam BM2. The elasticity-value calculating section 43 calculates an elasticity value (Young's modulus (in Pa: Pascal)) based on the velocity of propagation of the shear waves. It should be noted that only the velocity of propagation may be calculated without calculating the elasticity value.

According to the present embodiment, the T/R beamformer 3 transmits the first ultrasonic beam BM1 while steering it such that the first ultrasonic beam travels closest to and outside of the region R. The shape of the first ultrasonic beam BM1 is also defined to lie closest to and outside of the region R. Thus, shear waves generated by the first ultrasonic beam BM1 may be more reliably propagated to the region R while suppressing attenuation.

Next, a variation of the embodiment described above will be described. To begin with, a first variation will be described. At Step S2 described above, a pair of the first ultrasonic beams BM1-1, BM1-2 are transmitted, as shown in FIG. 10. The pair of the first ultrasonic beams BM1-1, BM1-2 are simultaneously transmitted to the outside of the region R and in the vicinity of both ends of the region R in the lateral direction.

In the present embodiment, again, the T/R beamformer 3 sets transmission parameters such that the beams BM1-1, BM1-2 travel closest to the region R based on information on the position of the region R, and transmits the first ultrasonic beams BM1-1, BM1-2. The first ultrasonic beams BM1-1, BM1-2 have a common focus F. The T/R beamformer 3 transmits the first ultrasonic beams BM1-1, BM1-2 by applying steering to them so that they intersect each other at the focus F.

In the first variation, shear waves generated by the first ultrasonic beam BM1-1 and those generated by the second ultrasonic beam BM1-2 are each detected by the second ultrasonic beam BM2 at Step S3 described above.

Next, a second variation will be described. At Step S2 described above, a pair of first ultrasonic beams BM1-1, BMI-2 are transmitted, as shown in FIG. 11. In particular, the T/R beamformer 3 sets transmission parameters such that the first ultrasonic beams BM1-1, BM1-2 are each transmitted to a position that lies in the vicinity of either end of the region R in the lateral direction and causes part of the first ultrasonic beams BM1-1, BM1-2 to be included in the region R, and transmits the pair of the first ultrasonic beams BM1-1, BM1-2. The configuration other than this feature is similar to the fist embodiment.

In the second variation, part of the first ultrasonic beams BM1-1, BM1-2 is included in the region R. The pair of the first ultrasonic beams BM1-1, BM1-2, however, are each transmitted in the vicinity of either end of the region R in the lateral direction, so that shear waves generated by one of the first ultrasonic beams BM may be propagated to a portion of the region R in which the other of the first ultrasonic beams is included, thus providing an elasticity image.

While the present invention has been described with reference to the embodiments, it will be easily recognized that the present invention may be practiced with several modifications without departing from the spirit and scope thereof. For example, the pair of the first ultrasonic beams BM1-1, BM1-2 may have respective different foci without having a common focus. In this case, the pair of the first ultrasonic beams BM1-1, BM1-2 intersect each other at a position other than the foci.

1. An ultrasonic diagnostic apparatus, comprising:
an ultrasonic probe for transmitting an ultrasonic beam to biological tissue in a subject;
a transmission control section for transmitting an ultrasonic beam for generating shear waves in said biological tissue from said ultrasonic probe to said biological tissue while applying steering to said beam; and
a region-defining section for defining a region in an ultrasonic image of said subject, wherein
said transmission control section transmits said ultrasonic beam while applying steering to said beam by setting transmission parameters such that said ultrasonic beam travels closest to and outside of said region based on information on a position of said region defined by said region-defining section.

2. The ultrasonic diagnostic apparatus as recited in claim 1, wherein said transmission control section adjusts a direction and a shape of said ultrasonic beam by setting said transmission parameters such that said ultrasonic beam travels closest to and outside of said region based on information on a position of said region defined by said region-defining section.

3. The ultrasonic diagnostic apparatus as recited in claim 1, wherein said transmission control section transmits one said ultrasonic beam or a pair of said ultrasonic beams.

4. An ultrasonic diagnostic apparatus comprising:
an ultrasonic probe for transmitting an ultrasonic beam to biological tissue in a subject;
a transmission control section for transmitting an ultrasonic beam for generating shear waves in said biological tissue from said ultrasonic probe to said biological tissue while applying steering to said beam; and
a region-defining section for defining a region in an ultrasonic image of said subject, wherein
said transmission control section transmits a pair of said ultrasonic beams while applying steering to said beams by setting transmission parameters such that said ultrasonic beams are each transmitted to a position that lies in the vicinity of either end of said region in a lateral direction and causes at least part of said ultrasonic beams to be included in said region based on information on a position of said region defined by said region-defining section.

5. The ultrasonic diagnostic apparatus as recited in claim 4 wherein said transmission control section adjusts a direction and a shape of said pair of ultrasonic beams by setting said transmission parameters such that said ultrasonic beams are each transmitted to a position that lies in the vicinity of either end of said region in a lateral direction and causes at least part of said ultrasonic beams to be included in said region based on information on a position of said region defined by said region-defining section.

6. The ultrasonic diagnostic apparatus as recited in claim 3, wherein said transmission control section simultaneously transmits said pair of ultrasonic beams.

7. The ultrasonic diagnostic apparatus as recited in claim 3, wherein said pair of ultrasonic beams have a common focus.

8. The ultrasonic diagnostic apparatus as recited in claim 1, wherein said transmission control section transmits a detecting ultrasonic beam for detecting shear waves generated by said ultrasonic beam(s).

9. The ultrasonic diagnostic apparatus as recited in claim 1, wherein a measurement-value calculating section for calculating a measurement value regarding elasticity of said biological tissue based on echo signals of said detecting ultrasonic beam.

10. The ultrasonic diagnostic apparatus as recited in claim 9, wherein a display section in which an elasticity image according to said measurement value is displayed in said region.

11. The ultrasonic diagnostic apparatus as recited in claim 9, wherein said measurement value is a velocity of propagation of said shear waves.

12. The ultrasonic diagnostic apparatus as recited in claim 9, wherein said measurement value is an elasticity value for biological tissue calculated based on the velocity of propagation of said shear waves.

13. The ultrasonic diagnostic apparatus as recited in claim 1, wherein said ultrasonic image is an ultrasonic image produced based on echo signals of an ultrasonic beam that is transmitted separately from said ultrasonic beam(s) for generating shear waves in said biological tissue.

14. The ultrasonic diagnostic apparatus as recited in claim 1, wherein said ultrasonic image is a B-mode image.

15. The ultrasonic diagnostic apparatus as recited in claim 1, wherein said ultrasonic probe is a convex probe.

16. The ultrasonic diagnostic apparatus as recited in claim 1, wherein said region-defining section defines said region based on an input by an operator.

17. An ultrasonic diagnostic apparatus comprising a processor, wherein the processor is configured to execute, by a program:
a transmission control function of transmitting an ultrasonic beam for generating shear waves in biological tissue in a subject from an ultrasonic probe to said biological tissue while applying steering to said beam; and
a region-defining function of defining a region in an ultrasonic image of said subject, wherein
said transmission control function is a function of transmitting said ultrasonic beam while applying steering to said beam by setting transmission parameters such that said ultrasonic beam travels closest to and outside of said region based on information on a position of said region defined by said region-defining function.

18. An ultrasonic diagnostic apparatus comprising a processor, wherein the processor is configured to execute, by a program:
a transmission control function of transmitting an ultrasonic beam for generating shear waves in biological tissue in a subject from an ultrasonic probe to said biological tissue while applying steering to said beam; and
a region-defining function of defining a region in an ultrasonic image of said subject, wherein
said transmission control function is a function of transmitting a pair of said ultrasonic beams while applying steering to said beams by setting transmission parameters such that said ultrasonic beams are each transmitted to a position that lies in the vicinity of either end of said region in a lateral direction and causes at least part of said ultrasonic beams to be included in said region based on information on a position of said region defined by said region-defining function.

19-20. (canceled)
